(19) United States
(12) Patent Application Publication
(10) Pub. No.: US 2025/0107747 A1
(43) Pub. Date: Apr. 3, 2025

(54) NEUROTECHNOLOGY SYSTEM AND METHOD FOR MONITORING SEIZURE DEVELOPMENT AND ASSESSING ANTI-SEIZURE DRUG RESPONSE

(71) Applicant: UNIVERSITY HEALTH NETWORK, Toronto (CA)

(72) Inventors: Nooshin BAHADOR, Toronto (CA); Liang ZHANG, Toronto (CA); Frances SKINNER, Toronto (CA); Milad LANKARANY, Toronto (CA)

(21) Appl. No.: 18/899,233

(22) Filed: Sep. 27, 2024

Related U.S. Application Data
(60) Provisional application No. 63/587,261, filed on Oct. 2, 2023.

(51) Int. Cl. A61B 5/00; G16H 20/10; G16H 50/20

(52) U.S. Cl. CPC A61B 5/4094; G16H 20/10; G16H 50/20

(57) ABSTRACT

According to an aspect, there is provided a computer implemented method and an epilepsy monitoring unit for diagnosis, investigation or treatment of seizures. The method includes identifying ictal-related chirp patterns from recordings of electrophysiological signals by determining onset and offset times of the ictal-related chirp patterns, characterizing the ictal-related chirp patterns, classifying spectro-temporal morphology of the ictal-related chirp patterns. According to an aspect, there is provided a computer implemented method for electrophysiological signals. The method including extracting a neuromarker from neural activities recorded as electrophysiological signals and characterizing ictal-related patterns in the electrophysiological signals during ictal discharge. The neuromarker is for assessment of progression of a seizure or efficacy of anti-seizure pharmacological agents.

### FIELD

The systems and methods described herein generally relate to the field of neurological disorders and more specifically to monitoring seizure development and anti-seizure drug response.

### INTRODUCTION

Neurological disorders such as epilepsy may be characterized by recurrent epileptic seizures. Despite the prevalence of such disorders, their underlying causes, methods of action, and progression are poorly understood. This lack of knowledge also makes developing and studying drugs to treat seizures or these neurological disorders difficult.

Improvements in the diagnosis, monitoring, and treatment for seizures and anti-seizure drug response are desirable.

### SUMMARY

Chirp characteristics of a patient's brain may indicate the likely progression of epilepsy in an individual. The systems and methods described herein can exploit distinct characteristics of chirp-like activities reflected in different states (early evoked discharge, late evoked discharge, spontaneous recurrent seizure, and a drug state). The systems and methods described herein make use of analyses of chirp-like activities to characterize disease progression and drug effects.

These systems and methods can be applied to, for example, tools to diagnose, monitor, treat, and investigate neurological disorders (e.g., wearable EEG) or to studying drug efficacy or mechanisms of action (e.g., in pharmaceutical trials). Such improvements may reduce the cost of healthcare and, in particular, reduce the number of hospital visits. These tools can also be implemented as a part of personalized care, for example, these methods can be personalized to a patient and treatment can be tailored to the patient's needs.

According to an aspect, there is provided a computer implemented method. The method includes identifying ictal-related chirp patterns from recordings of electrophysiological signals by determining onset and offset times of the ictal-related chirp patterns, characterizing the ictal-related chirp patterns, classifying spectro-temporal morphology of the ictal-related chirp patterns.

In some embodiments, determining the onset and offset times of the ictal-related chirp patterns includes computing a smoothed power ratio trajectory of defined high to low frequency bands over time, applying a threshold to the smoothed power ratio trajectory, identifying instances where the power ratio trajectory exceeds the threshold, confirming that the identified event is statistically significant, defining chirp onset as the moment when the power ratio trajectory significantly exceeds threshold, defining chirp offset as the moment when the power ratio trajectory significantly falls below threshold, and fine-tuning the defined range of high and low frequency bands.

In some embodiments, high-frequency range is set between 10 Hz and 22 Hz, and low-frequency range is set between 1 Hz and 10 Hz.

In some embodiments, the method further includes tracking chirp morphology by extracting a dominant frequency ridge curve from a time-frequency representation using a penalized forward-backward greedy algorithm between onset and offset times of the ictal-related chirp patterns with customizable parameters to penalize frequency changes and determine a number of ridges.

In some embodiments, characterizing the ictal-related chirp patterns includes determining temporal and spectral characteristics by analyzing features comprising duration of chirp, median frequency of chirp, and chirp onset.

In some embodiments, classifying the spectro-temporal morphology of the ictal-related chirp patterns comprises categorizing the spectro-temporal morphology into a type based on the evolution of frequency characteristics over time.

In some embodiments, generating the data about the chirp morphology includes statistical testing including one-way Analysis of Variance (ANOVA) and Tukey's test to identify specific pairs of conditions displaying significant differences.

In some embodiments, the method further includes extracting the time-frequency representation by spectrogram calculation of segmented the electrophysiological signal, applying windowing functions, and performing Discrete Fourier Transform to reveal spectral evolution of the electrophysiological signal over time.

In some embodiments, determining the onset and offset of chirp includes analyzing a power ratio of specific frequency bands and identifying chirp events based on a threshold.

In some embodiments, the method further includes generating the data about the chirp morphology by determining characteristics of chirp-like activities that reflect different subject states comprising early evoked discharge, late evoked discharge, spontaneous recurrent seizure, and a drug state.

In some embodiments, the electrophysiological signals are selected from the group of EEG, intracranial EEG, and Local Field Potentials signals.

In some embodiments, a chirp is an electrophysiological signal whose frequency changes over time.

In some embodiments, the method further includes characterizing at least one of progression of a disease and effect of a drug based in part on the ictal-related chirp patterns.

In some embodiments, the method further includes elucidating a mechanism of action of a drug based in part on the ictal-related chirp patterns.

In some embodiments, classifying the spectro-temporal morphology of the ictal-related chirp patterns includes categorizing the spectro-temporal morphology into one of Type 1-5. Type 1 exhibits a decline in frequency with a semi-linear trend. Type 2 exhibits a stepped increment in frequency over time. Type 3 exhibits asymmetric peak-structured frequency variations. Type 4 exhibits symmetric peak-structured frequency changes. Type 5 exhibits a rapid initial increase in frequency followed by a stable frequency profile over time.

In some embodiments, classifying the spectro-temporal morphology of the ictal-related chirp patterns includes determining that the spectro-temporal morphology of the ictal-related chirp patterns occur in a cyclic manner.

In some embodiments, the method further includes determining whether the ictal-related chirp patterns arise from a spontaneous ictal event or an evoked discharged based on timing of the chirp within the ictal event.

In some embodiments, the method further includes verifying the spectro-temporal morphology classification of the ictal-related chirp patterns by comparing the spectro-temporal morphology classification with a spectro-temporal morphology classification of ictal-related chirp patterns arising from another brain region.

According to an aspect, there is provided an epilepsy monitoring unit for diagnosis, investigation or treatment of seizures including one or more sensors for capturing electrophysiological signals, a processing system that includes one or more processors and one or more memories coupled with the one or more processors, the one or more memories storing recordings of electrophysiological signals. The processing system configured to cause monitoring unit to identify ictal-related chirp patterns from recordings of electrophysiological signals by determining onset and offset times of the Ictal-related chirp patterns, characterize the ictal-related chirp patterns, and classify chirp morphology.

According to an aspect, there is provided a computer implemented method for electrophysiological signals. The method including extracting a neuromarker from neural activities recorded as electrophysiological signals and characterizing ictal-related patterns in the electrophysiological signals during ictal discharge. The neuromarker is for assessment of progression of a seizure or efficacy of anti-seizure pharmacological agents.

In some embodiments, the neuromarker comprises a plurality of features derived from chirp-like patterns in the electrophysiological signals, the plurality of features comprising morphology of a chirp, duration of the chirp, onset time, frequency band and power distribution.

In some embodiments, the method further includes generating neuromarker-based evaluations or developing a personalized treatment plan to improve seizure control.

Many further features and combinations thereof concerning embodiments described herein will appear to those skilled in the art following a reading of the instant disclosure.

### DESCRIPTION OF THE FIGURES

In the figures,

FIG. 1 illustrates a method to infer chirp-like patterns from EEG recordings, characterize those patterns, and classify them by their morphologies, according to some embodiments.

FIG. 2 illustrates a process flow for the detection of chirp onset and offset times, according to some embodiments.

FIG. 3 illustrates a spectrogram and a corresponding extracted ictal-related chirp, according to some embodiments.

FIG. 4 illustrates spectro-temporal morphology of various types of ictal chirps, according to some embodiments.

FIG. 5 illustrates a method of analyzing chirp patterns, according to some embodiments.

FIG. 6 illustrates a schematic diagram of an epilepsy monitoring unit, according to some embodiments.

FIG. 7 illustrates a method using chirp patterns as a neuromarker, according to some embodiments.

FIG. 8A illustrates a 3D visualization of a sample ictal discharge chirp with asymmetric peak-structured frequency variation over time, according to some embodiments.

FIG. 8B illustrates the recording and 2D time-frequency representation accompanying the visualization of FIG. 8A, according to some embodiments.

FIG. 9 illustrates chirps exhibiting harmonic relationships, according to some embodiments.

FIG. 10 illustrates characteristics of a chirp pre, during, and post drug administration, according to some embodiments.

FIG. 11 illustrates further characteristics of a chirp pre, during, and post the drug administration of FIG. 10, according to some embodiments.

FIG. 12 illustrates changes in mean values and confidence intervals for chirp-like pattern characteristics as epilepsy progresses from the initial evoked state to a drug state, according to some embodiments.

FIG. 13 illustrates changes in chirp median frequency from early to late evoked discharge, according to some embodiments.

FIG. 14A illustrates the temporal relationship between chirp occurrence and ictal termination for an evoked discharge, according to some embodiments.

FIG. 14B illustrates the temporal relationship between chirp occurrence and ictal termination for a spontaneous recurrent seizure ictal discharge, according to some embodiments.

FIG. 15A illustrates concurrent chirp patterns during spontaneous recurrent seizure from the ictal discharge in the hippocampus, according to some embodiments.

FIG. 15B illustrates concurrent chirp patterns during spontaneous recurrent seizure from the ictal discharge in the piriform cortex, according to some embodiments.

FIG. 16 illustrates a schematic diagram of computing device, according to some embodiments.

FIG. 17 illustrates a schematic diagram of a system with a computing device and a sensor, according to some embodiments.

FIG. 18 illustrates a schematic diagram of a system with a computing device and a visual output, according to some embodiments.

FIG. 19 illustrates a schematic diagram of a system with a computing device and a server, according to some embodiments.

### DETAILED DESCRIPTION

The epileptic brain may generate chirp-like activities, yet understanding the evolution of these chirp-like patterns across various stages of epilepsy, from the initial phase to a medicated state, remains unclear. Achieving a consistent pattern in clinical data is challenging. It is not feasible to have a large number of seizures for each patient, and obtaining long-term intracranial recordings of human subject presents logistical difficulties.

An epileptic seizure can involve of a series of distinct phases over time, including initiation, maintenance, and termination that can vary widely in their characteristics and duration. Some seizures are brief, while others can be more prolonged. The progression of epilepsy itself can depend on various factors, including the seizure frequency, seizure types, age of onset, underlying cause, etc. Some types of epilepsy may progress over time, with varying degrees of structural damage observed in individuals based on the duration of the condition, although the heterogeneity of individuals' responses to this neurological disease can result in diverse progression rates, ranging from aggressive to slower or more stable courses.

In fields like optics and telecommunications, a “chirp” refers to a signal whose frequency changes over time and can emerge when a pulse undergoes manipulation during its propagation through a medium with dispersion properties. Chirps can be found in signals across a wide range of biological and physical phenomena, including bird songs, insect communication, and radar systems. The epileptic brain can also produce chirp-like patterns.

These chirp-like patterns may exhibit time-frequency representations within seizure data. The timing and frequency dynamics of these chirps may be more difficult to study. The reported timing of chirp occurrences differs, with some indicating onset of ictal discharge, others during transition from interictal to ictal states, and some even prior to seizure. The frequency dynamics of chirps in these studies may also be different, including, for example, linear frequency decrease, upward-downward trend, and harmonic structures. Chirp characteristics of a patient's brain may indicate the likely progression of epilepsy in an individual.

Described herein are methods for the reliable characterization of chirp-like activities during ictal events. Using these methods, activities across various states of disease progression and drug application can be characterized with extracted features showing statistically significant differences among the different states. Described herein is a method to reliably infer chirp-like patterns from EEG recordings, characterize those patterns, and classify them by their morphologies. Though the following discussion is primarily in the context of one type of epilepsy, other types of epilepsy as well as seizures in other neurological disease states may also be characterized using the methods described herein.

Described herein is a reliable neurotechnology for extracting, analyzing and characterizing chirp-like patterns from ictal discharge signals. The neurotechnology can detect the onset and offset of chirp events, extract its time-frequency trajectory, and provide detailed information about the morphology of the chirp, its duration, onset time, frequency band and power distribution. Embodiments of the neurotechnology have been evaluated using a novel experimental data set of epilepsy kindling in mice to show that distinct characteristics of chirp-like activities reflect different states (early evoked discharge, late evoked discharge, spontaneous recurrent seizure, and a drug state). The detailed analyses of chirp-like activities provided by the neurotechnology can characterize epilepsy progression and drug effects.

In some embodiments, excitatory-inhibitory network models may be used to obtain mechanistic insights into the chirp phenomenon and its frequency variations by, for example, taking advantage of observed changes such as inhibitory (GABAA) enhancing drugs like lorazepam.

### General Method to Characterize Chirp-Like Patterns.

The methods used herein for data analysis can include chirp identification, ridge extraction and statistical testing. For time-frequency representation extraction, spectrogram calculation can involve segmenting the signal, applying windowing functions, and performing a Discrete Fourier Transform to reveal the signal's spectral evolution over time. Time-frequency ridges, representing dominant frequency components, can be extracted using a forward-backward greedy algorithm with customizable parameters to penalize frequency changes and determine the number of ridges. For extracting ridge, the onset and offset of chirp can be determined by analyzing power ratio of specific frequency bands and identifying chirp event based on a threshold. Lastly, statistical tests, including one-way ANOVA and Tukey's test, can be applied to analyze differences among distinct conditions.

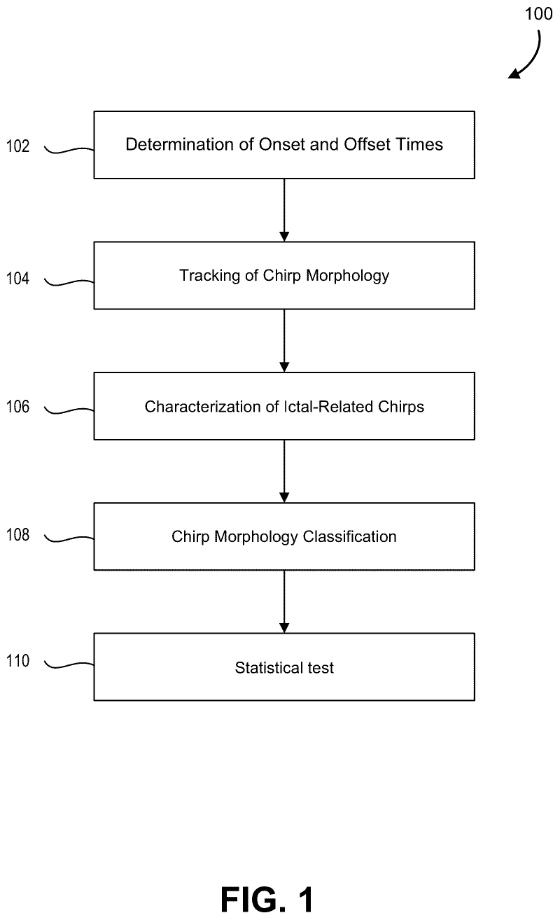

Methods, such as those embodied by FIG. 1, use a combination of features derived from chirp-like patterns in EEG signals as a neuromarker that enables assessing the progression of seizure and efficacy of anti-seizure pharmacological agents. Features include, for example, the morphology of the chirp, its duration, onset time, frequency band and power distribution.

The systems and methods described herein can consistently extract this neuromarker from neural activities (recorded from electrophysiological signals like EEG, intracranial EEG, and Local Field Potentials) and reliably characterize chirp-like activities during ictal discharge.

FIG. 1 illustrates a method 100 to infer chirp-like patterns from EEG recordings, characterize those patterns, and classify them by their morphologies, according to some embodiments.

The method 100 can comprise the steps of determining the onset and offset times of ictal-related chirps (block 102), tracking of chirp morphology (block 104), characterizing the ictal-related chirps (block 106), classifying chirp morphology (block 108), and statistical tests (block 110). In some embodiments, once method 100 has been carried out, then the ictal-related chirps of a patient may be analyzed using similar methodologies (e.g., one or more steps of method 100) and insights of the patient's ictal events may be ascertained (e.g., seizure or disease type, disease progression, drug efficacy, etc.). Furthermore, characteristics of the patient's ictal-related chirps can be stored and compared to assess patient progression (e.g., comparing a patient's chirp characteristics to their past characteristics).

### Determination of Onset and Offset Times for Ictal-Related Chirps (Block 102).

The determination of onset and offset times of Ictal-related chirps (block 102) can involve the following steps: a) computing a smoothed (e.g., using a moving average) power ratio trajectory of pre-defined high to low frequency bands over time (high-frequency range can be set between, for example, 10 Hz and 22 Hz, and low-frequency range can be set between, for example, 1 Hz and 10 Hz; these frequency bands can be adapted and tuned depending on the subject); b) applying a threshold of, for example, 1.3-1.9 or 1.5 to the smoothed power ratio trajectory (the threshold can be adjusted based on characteristics of the subject (e.g. range of frequencies vary depending on the subject, adjust based on baseline before drug administration), the threshold may be linked to a range of frequency bands from step a)); c) identifying instances (e.g., using statistical measures) where the power ratio trajectory exceeds the threshold; d) confirming the significance of the identified event through statistical analysis; e) defining chirp onset as, for example, the moment when the power ratio trajectory significantly exceeds the threshold; f) defining chirp offset as, for example, the moment when the power ratio trajectory significantly falls below the threshold; g) fine-tuning the pre-defined range of high and low frequency bands through visual assessment (e.g., with respect to the baseline). In some embodiments, the instantaneous frequency can be tracked to see if there is a dominant frequency for the dataset. This may provide the range for the frequency.

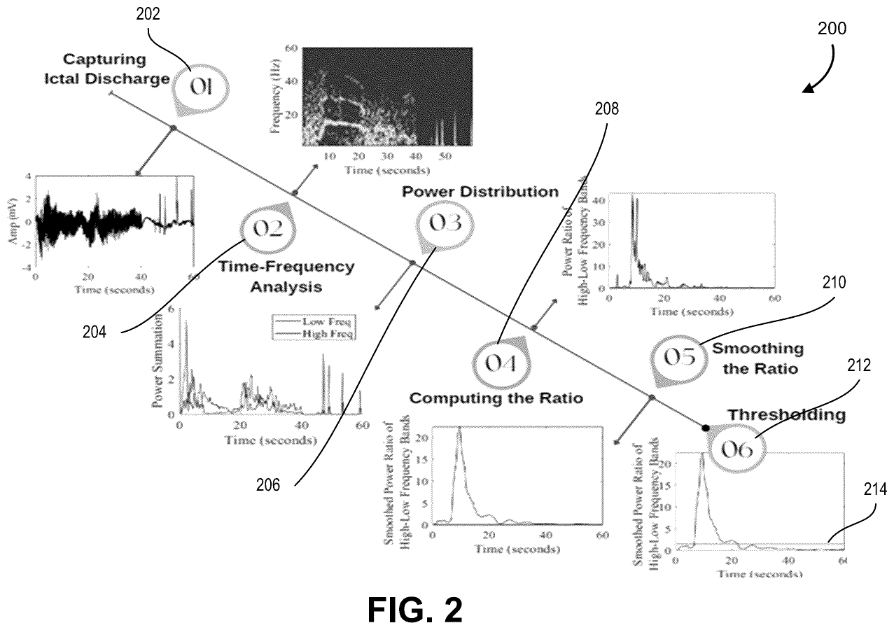

FIG. 2 illustrates a process flow 200 for the detection of chirp onset and offset times infer chirp-like patterns from EEG recordings, according to some embodiments.

Automatic detection of chirp onset and offset times can involve capturing ictal discharge (step 202), conducting time-frequency analysis (step 204), calculating power distribution in pre-defined bands (step 206), determining the high-to low-frequency power ratio (step 208), applying smoothing to the ratio trajectory (step 210), and setting thresholds (step 212). When evaluating the smoothed power ratio of high-low frequency bands over time, a threshold of, for example, 1.5 (line 214), the high-frequency range being, for example, 10 Hz to 22 Hz, and the low-frequency range spanning from, for example, 1 Hz to 10 Hz can be considered. The smoothed power ratio trajectory can be divided into two distinct groups: values exceeding the threshold and values equal to or below the threshold. Utilizing a two-sample t-test, a comparison of the means of these two groups may reveal a very low p-value (e.g., p<1e-79) in experimental results, indicating a difference between the groups. The chirp's initiation and termination can be defined as instances when the smoothed power ratio exceeds the specified threshold.

### Tracking of Chirp Morphology (Block 104).

After determining the onset and offset times, chirp morphology tracking can then be performed by, for example, extracting the dominant frequency ridge curve from the spectrogram (time-frequency representation) using a penalized forward-backward greedy algorithm between the determined onset and offset times of ictal-related chirp.

### Characterization of Ictal-Related Chirps (Block 106).

The automated characterization of ictal-related chirps can involve analyzing key features such as the duration of chirp, median frequency of chirp, and chirp onset (distance from ictal discharge onset) to better understand the temporal and spectral characteristics. Additional features can also be extracted from chirps, including, for example, “Rise time” in spectro-temporal pattern of upward chirps, “Fall time” in spectro-temporal pattern of downward chirp, “curvature” of chirp's spectro-temporal pattern, “symmetry” of chirp's spectro-temporal pattern, “asymmetry” of chirp's spectro-temporal pattern, “slope” of linear chirp's spectro-temporal pattern, “Area under the curve” of chirp's spectro-temporal pattern, “Peak” of chirp's spectro-temporal pattern, “sharpness” of chirp's spectro-temporal pattern, “smoothness” of chirp's spectro-temporal pattern, “irregularity” of chirp's spectro-temporal pattern, “Median frequency of chirp's harmonic” of chirp's spectro-temporal pattern, “min and max frequency” of chirp, Theta-gamma coupling between onset and offset of chirp. These chirp-derived features may provide valuable insight. These chirp-derived features may further provide insights based on how they change over time and how they change in different states. Further features can include, for example, couple's oscillations.

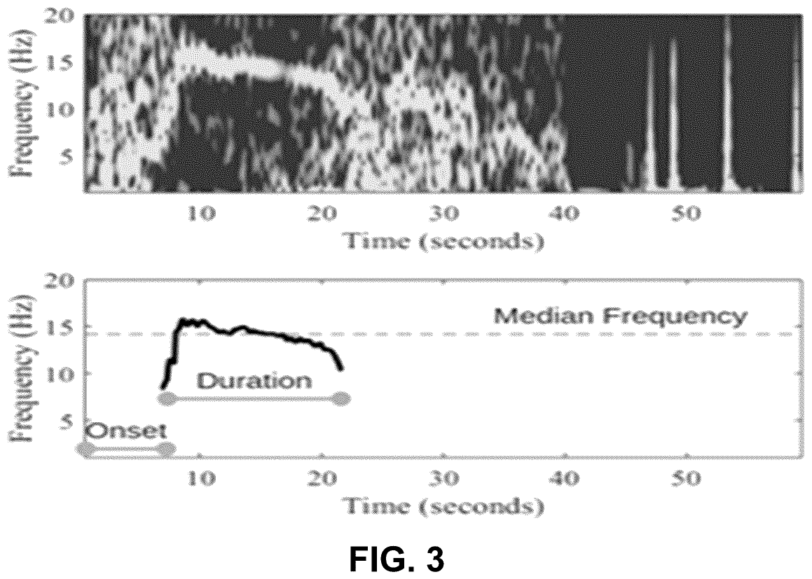

FIG. 3 illustrates a spectrogram and a corresponding extracted ictal-related chirp, according to some embodiments. Key features have been highlighted including the chirp's duration, median frequency, and chirp onset (distance from ictal discharge onset).

### Chirp Morphology Classification (Block 108).

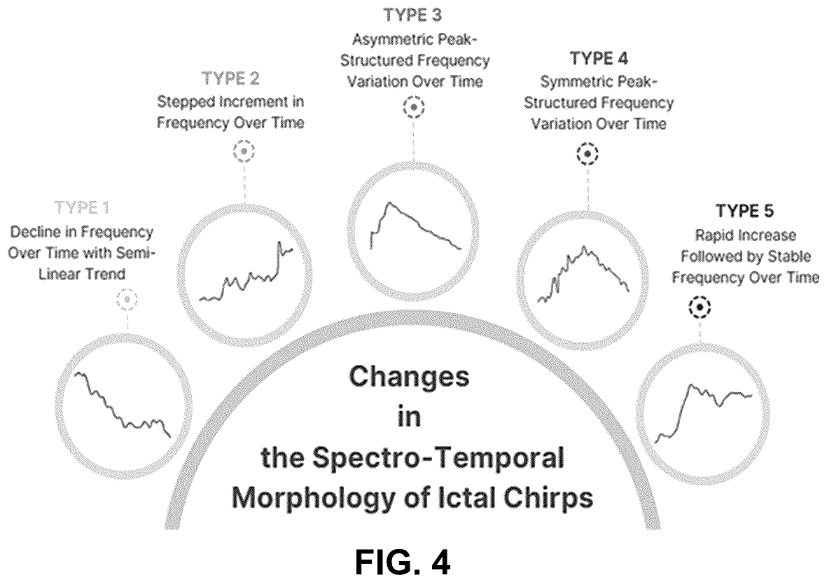

FIG. 4 illustrates spectro-temporal morphology of various types of ictal chirps, according to some embodiments.

Chirp type (the morphology type of chirp) may encode dynamical properties of the seizure and/or underlying mechanisms responsible for seizure. The chirp type may further be used to ascertain the mechanism and cause behind the seizure (e.g., the “how” and “why”) potentially at both cellular and network levels. The types can potentially be used to decipher different states and drug effects.

The spectro-temporal morphology of ictal-related chirps can be categorized into five distinct example types based on the evolution of their frequency characteristics over time. Type 1 may exhibit a decline in frequency with a semi-linear trend. Type 2 may display a stepped increment in frequency over time. Type 3 may feature asymmetric peak-structured frequency variations, while Type 4 may showcase symmetric peak-structured frequency changes. Type 5 may be characterized by a rapid initial increase in frequency followed by a stable frequency profile over time. These patterns may be the ones most frequently observed in experimental results described below. They may be determined based on visual assessment. Other types of morphology may be conceived, including additional sub-types.

Insights may be ascertained by determining which type is present during an ictal event. Insights may also be gleaned should the type change from baseline (e.g., after drug administration to assess drug efficacy or over time to assess disease progression).

### Statistical Test (Block 110).

Statistical tests can be applied to investigate differences among distinct conditions to identify relevant features for further processing. Initially, a one-way Analysis of Variance (ANOVA) may be conducted. Subsequently, Tukey's test may be employed to pinpoint specific pairs of conditions displaying significant differences. These tests may be run on, for example, large populations to aid in the identification of possible features to analyze on a patient by patient basis. In some embodiments, the populations may be patient populations. In some embodiments, the populations may be animal populations. In some embodiments, insights determined by analyzing animal populations may be applicable to human populations. The advantage of using animal populations is that animal models may produce a greater frequency of ictal events for analysis. This may be particularly important where there is limited ictal data for human patients. These features may be determine based on their statistical significance. In some embodiments, the statistical significance may be a p-value of less than 0.05. Other thresholds may also be implemented. The statistical analysis may be used to determine whether two states (or two groups) are significantly different. For instance, to monitor the development of epilepsy from its initial evoked state to its later stages, significant changes in the derived characteristics from the chirp signal can be sought as the state transitions from the early to the late states.

Statistical tests conducted on rodent data aided in identifying a set of chirp-derived features that may be relevant and can characterize different states over time. This may help elucidate the significance of these features and what to look for.

Once a model has been developed it can be used to assess the ictal events of individual patients and differentiate between different states which may provide insights into ictal event type, disease progression, drug efficacy, or other phenomena.

### Uses.

Once the ictal chirp has been analyzed by the above method it can be used as a reliable neuromarker for both evoked and spontaneous recurrent seizure events. The morphology and the features extracted help understand what stage of, for example, epilepsy, the subject is in (earlier or advanced). Monitoring changes in chirp characteristics can allow progression tracking (e.g., to see whether the condition is aggressive or gradual).

In some embodiments, the chirp-like patterns can be used to characterize disease progression and drug effects. For example, by assessing the chirp characteristics before and after drug administration, differences (or lack thereof) between the two can provide insights as to whether the drug influences the ictal discharge. In some embodiments, monitoring changes in chirp characteristics over time after drug administration can provide insights for drug metabolism and dosing (e.g., repeat doses can be timed as the chirp characteristics return (or begin to return) to pre-administration levels). In some embodiments, changes in chirp characteristics after drug administration can help provide insights into how quickly the drug is metabolized.

In some embodiments, the chirps can provide support for the mechanism of action for drugs provided to patient. If the same chirp characteristics are observed after/during drug administration as were observed before, it may suggest that the drug may not suppress the ictal discharge and has no effect on them. However, if characteristic of the chirp changes (e.g., increased duration) during or after drug administration, then it may indicate that the drug may enhance or suppress ictal rhythmic discharges. Additionally, by monitoring when the chirp returns to its baseline characteristics after recovery from the drug, insights into the duration of this particular drug's effects can be gained. Furthermore, the characteristics that change may provide insights into the mechanism of action (e.g., if the duration of the chirp changes, but the smoothness does not). Furthermore, changes in the type of chirp may also provide insight into a drug's mechanism of action and the mechanism of the seizure itself.

In some embodiments, the frequencies of the individual chips may exhibit a harmonic relationship. Harmonics may indicate the involvement of multiple pathways for the migration of seizures as they propagate through the brain or could indicate the presence of multiple clusters of neurons involved in the seizure.

In some embodiments, different types of spectro-temporal morphology of ictal chirps occur in a cyclic manner. For example, a chirp may initiate as, for example, chirp Type 3, subsequently transition to, for example, Type 4, undergo another transformation into, for example, Type 5, then revert to, for example, Type 4 before transitioning back to, for example, Type 3, and then shift to, for example, Type 5. This may suggest a potentially cyclic occurrence of different types of spectro-temporal morphology in ictal chirps. The cyclic patterns of types may provide information about the disease, the drug, or the specific mechanism of action.

In some embodiments, Ictal-Related Chirps start earlier after many stimulations (i.e., late evoked states). Initially, it takes longer to observe the chirp at the early stimulation stage. As more stimulations occur, the system somehow becomes more facilitative in generating this activity. As such, it may be possible to use the chirp as an indicator of the stage of the neurological disease (e.g., epilepsy) and distinguish between early or advanced stages.

In some embodiments, chirps can be differentiated between evoked chirps and spontaneous ictal events. For example, the chirps may occurs just prior to termination for evoked discharges while chirps may occur clearly within the maintenance phase (middle) for spontaneous ictal events. In other words, evoked discharges may terminate abruptly following the chirp, while spontaneous ones do so less distinctly. The chirps of evoked discharges may be useful in ascertaining insights for the chirps found in spontaneous discharges.

In some embodiments, comparable chirp patterns can be detected in different brain regions within the same animal during an episode of spontaneous recurrent seizure. In some embodiments, signals from different brain regions may be used to ascertain insights into a patient's seizure. Experimental results (described in greater detail below) show that ictal events can give rise to phenomena in different brain regions. In some embodiments, signals recorded from different brain regions during an ictal event can be used to validate the signals (e.g., to track the ictal event across different brain regions at once). In some embodiments, the differences (or lack thereof) in chirp characteristics or even chirp type across different brain regions may reveal insights into the underlying seizure mechanism or other insights.

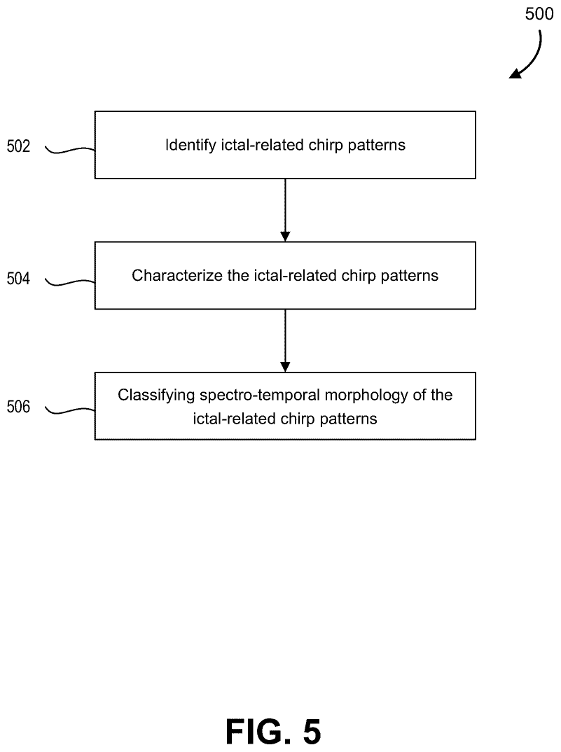

FIG. 5 illustrates a method 500 of analyzing chirp patterns, according to some embodiments.

According to an aspect, there is provided a computer implemented method 500. The method 500 includes identifying ictal-related chirp patterns from recordings of electrophysiological signals by determining onset and offset times of the ictal-related chirp patterns (502), characterizing the ictal-related chirp patterns (504), classifying spectro-temporal morphology of the ictal-related chirp patterns (506).

In some embodiments, determining the onset and offset times of the ictal-related chirp patterns includes computing a smoothed power ratio trajectory of defined high to low frequency bands over time, applying a threshold to the smoothed power ratio trajectory, identifying instances where the power ratio trajectory exceeds the threshold, confirming that the identified event is statistically significant, defining chirp onset as the moment when the power ratio trajectory significantly exceeds threshold, defining chirp offset as the moment when the power ratio trajectory significantly falls below threshold, and fine-tuning the defined range of high and low frequency bands.

In some embodiments, high-frequency range is set between 10 Hz and 22 Hz, and low-frequency range is set between 1 Hz and 10 Hz.

In some embodiments, the method further includes tracking chirp morphology by extracting a dominant frequency ridge curve from a time-frequency representation using a penalized forward-backward greedy algorithm between onset and offset times of the ictal-related chirp patterns with customizable parameters to penalize frequency changes and determine a number of ridges.

In some embodiments, characterizing the ictal-related chirp patterns (504) includes determining temporal and spectral characteristics by analyzing features comprising duration of chirp, median frequency of chirp, and chirp onset.

In some embodiments, classifying the spectro-temporal morphology of the ictal-related chirp patterns (506) includes categorizing the spectro-temporal morphology into a type based on the evolution of frequency characteristics over time.

In some embodiments, generating the data about the chirp morphology includes statistical testing including one-way Analysis of Variance (ANOVA) and Tukey's test to identify specific pairs of conditions displaying significant differences.

In some embodiments, the method further includes extracting the time-frequency representation by spectrogram calculation of segmented the electrophysiological signal, applying windowing functions, and performing Discrete Fourier Transform to reveal spectral evolution of the electrophysiological signal over time.

In some embodiments, determining the onset and offset of chirp includes analyzing a power ratio of specific frequency bands and identifying chirp events based on a threshold.

In some embodiments, the method further includes generating the data about the chirp morphology by determining characteristics of chirp-like activities that reflect different subject states comprising early evoked discharge, late evoked discharge, spontaneous recurrent seizure, and a drug state.

In some embodiments, the electrophysiological signals are selected from the group of EEG, intracranial EEG, and Local Field Potentials signals.

In some embodiments, a chirp is an electrophysiological signal whose frequency changes over time.

In some embodiments, the method further includes characterizing at least one of progression of a disease and effect of a drug based in part on the ictal-related chirp patterns.

In some embodiments, the method further includes elucidating a mechanism of action of a drug based in part on the ictal-related chirp patterns.

In some embodiments, classifying the spectro-temporal morphology of the ictal-related chirp patterns (506) includes categorizing the spectro-temporal morphology into one of Type 1-5. Type 1 exhibits a decline in frequency with a semi-linear trend. Type 2 exhibits a stepped increment in frequency over time. Type 3 exhibits asymmetric peak-structured frequency variations. Type 4 exhibits symmetric peak-structured frequency changes. Type 5 exhibits a rapid initial increase in frequency followed by a stable frequency profile over time.

In some embodiments, classifying the spectro-temporal morphology of the ictal-related chirp patterns (506) includes determining that the spectro-temporal morphology of the ictal-related chirp patterns occur in a cyclic manner.

In some embodiments, the method further includes determining whether the ictal-related chirp patterns arise from a spontaneous ictal event or an evoked discharged based on timing of the chirp within the ictal event.

In some embodiments, the method further includes verifying the spectro-temporal morphology classification of the ictal-related chirp patterns by comparing the spectro-temporal morphology classification with a spectro-temporal morphology classification of ictal-related chirp patterns arising from another brain region.

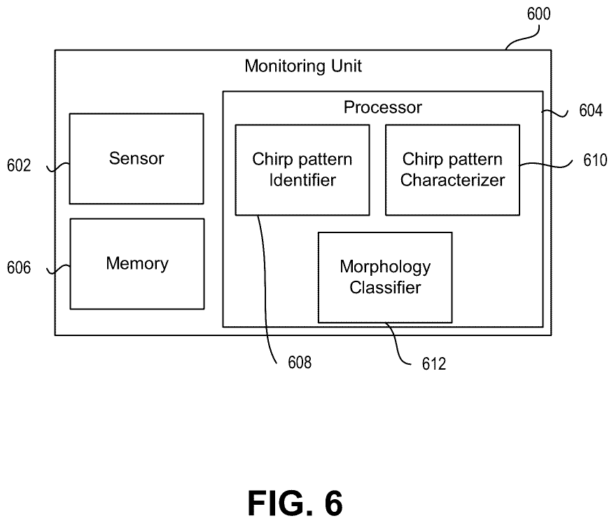

FIG. 6 illustrates a schematic diagram of an epilepsy monitoring unit 600, according to some embodiments.

According to an aspect, there is provided an epilepsy monitoring unit 600 for diagnosis, investigation or treatment of seizures including one or more sensors 602 for capturing electrophysiological signals, a processing system that includes one or more processors 604 and one or more memories 606 coupled with the one or more processors 604, the one or more memories 606 storing recordings of electrophysiological signals. The processing system configured to cause monitoring unit to identify ictal-related chirp patterns from recordings of electrophysiological signals by determining onset and offset times of the Ictal-related chirp patterns using chirp pattern identifier 608, characterize the ictal-related chirp patterns using chirp pattern characterizer 608, and classify chirp morphology using morphology classifier 612.

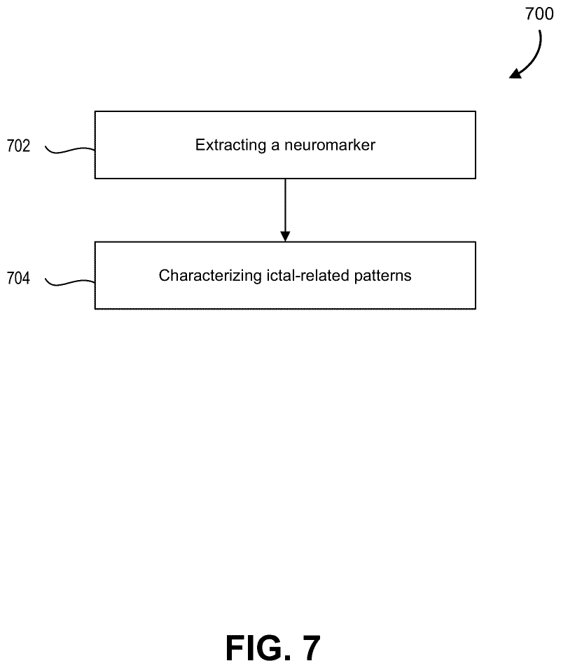

FIG. 7 illustrates a method 700 using chirp patterns as a neuromarker, according to some embodiments.

According to an aspect, there is provided a computer implemented method 700 for electrophysiological signals. The method including extracting a neuromarker from neural activities recorded as electrophysiological signals (702) and characterizing ictal-related patterns in the electrophysiological signals during ictal discharge (704). The neuromarker is for assessment of progression of a seizure or efficacy of anti-seizure pharmacological agents.

In some embodiments, the neuromarker comprises a plurality of features derived from chirp-like patterns in the electrophysiological signals, the plurality of features comprising morphology of a chirp, duration of the chirp, onset time, frequency band and power distribution.

In some embodiments, the method 700 further includes generating neuromarker-based evaluations or developing a personalized treatment plan to improve seizure control.

### Example Experimental Results

The following discussion describes example experimental results of using ictal-related chirps as a bio-marker for monitoring seizure development and drug response. The following discussion is provided by way of example only and is not intended to limit the scope of the concepts described herein.

Chirps may be an inherent characteristic of ictal discharge, which persists regardless of whether it is evoked or spontaneous. Furthermore, these chirps may not be exclusive to the hippocampus and may occur in the piriform cortex. These chirps may also provide support for the action mechanism of drugs. For example, Lorazepam, classified as a GABAA enhancer, may extend the duration of the ictal chirp, reduce the median frequency of chirps, and prolong the time before chirp onset. Additionally, significant changes in the frequency and duration of chirp may be observed when transitioning from early evoked to late evoked ictal events.

The potential count of ictal discharges for a human subject during a clinical trial may be limited to a small number (fewer than four) over the span of one month in, for example, an optimal scenario. The determination of surgery candidates may therefore be reliant on observing these few occurrences of ictal discharges. Alternatively, animal models may show an average of two discharges per day. With a consistently high daily discharge frequency over the course of several months, distinct chirp characteristics can be extracted from this substantial volume of ictal discharges. This may be of clinical significance as distinct characteristics could represent different states and/or different levels of disease progression.

The ability to fully characterize the chirp may be limited due to insufficiency of ictal discharges data. Animal models can generate chirp data through long-term recording. The quantity of available chirps for this analysis may be substantially greater by orders of magnitude. This can enable continuously tracking the dynamics of the chirp pattern over time. Described herein is reliable characterization of ictal-related chirps.

The systems and methods described herein exploit the chirp as a reliable measure for analyzing disease progression and assessing the impact of clinically administered anti-epileptic drugs (e.g., Lorazepam, the cleanest and best-defined in terms of its action mechanism).

### Materials and Methods—Detailed Description of Experiments—Animals.

C57 black mice (C57BL/6N, male, 11-13 months-old) were used. Mice were kept in standard cage condition and maintained at a temperature of 22-23° C. and with a 12-hour light on/off cycle (light-on starting at 6:00 am).

### Materials and Methods—Detailed Description of Experiments—Electrode Implantation and Hippocampal Kindling.

All electrodes were made of polyamide-insulated stainless-steel wires (110 μm outer diameter). Electrode implantation was under isofluorane anesthesia. Each mouse was implanted with two pairs of twisted-wire bipolar electrodes. One pair of electrodes was positioned to the hippocampal CA3 region for kindling stimulation and local recordings (bregma-2.5 mm, lateral 3.0 mm, and depth 3.0 mm), and another pair positioned to an ipsilateral or contralateral site. The latter included the contralateral hippocampal CA3, parietal cortex, piriform cortex and entorhinal cortex. A reference electrode was positioned to a frontal area. The putative tip locations of implanted electrodes were determined later in brain histological experiments if suitable.

A train of stimuli at 60 Hz for 2 seconds was used for hippocampal kindling. Kindling stimuli were applied twice daily and >5 hours apart. Each stimulation episode lasted for a few minutes while the mouse was placed in a glass container for EEG-video monitoring. Control mice experienced twice daily handlings for 60 days.

### Materials and Methods—Detailed Description of Experiments—EEG Recordings and Continuous EEG-Video Monitoring.

Local differential recordings through the twisted-wire bipolar electrodes were used to monitor evoked responses and spontaneous EEG activities. Evoked and spontaneous EEG signals were collected using two-channel or one-channel microelectrode AC amplifiers with extended head-stages. These amplifiers were set with an input frequency band of 0.1-1,000 Hz and an amplification gain of 1,000. A build-in notch filter at 60+3 Hz were used in some experiments. Amplifier output signals were digitized at 5,000 Hz. Data acquisition, storage and analyses were done using pClamp software.

Continuous 24-hour EEG-video monitoring in free-moving mice was done in a modified cage as previously described. Dim lighting was used for video monitoring during the light-off period. A cursor auto-click program was used to operate EEG and video recordings and save data every 2 hours. EEG and video data were collected roughly 24 hours daily and for up to 6 consecutive days per session.

Individual mice underwent EEG-video monitoring for 24 hours after about 80, 100, 120 and 140 kindling stimulations. If ≥2 spontaneous recurrent seizure (SRS) events were observed in the 24-hour monitoring, no further kindling stimulation was applied and EEG-video continued for up 6 days to assess SRS in the early phase post kindling. EEG-video monitoring up to 6 consecutive days was resumed in some mice 6-12 weeks later to assess SRS in the late phase of post kindling.

### Materials and Methods—Detailed Description of Experiments—Seizure Analysis.

Evoked ADs and spontaneous discharges were recognized by repetitive spikes with simple and/or complex waveforms, amplitudes approximately 2 times the background signals and durations of ≥10 seconds. Motor seizures were scored using the Racine scale modified for mice. Briefly, stage 0, 1 and 2 motor seizures were recognized by behavioral arrest, chewing/facial movement, and head nodding/unilateral forelimb clonus; stage 3, 4 and 5 motor seizures were recognized by bilateral forelimb clonus, rearing, and falling, respectively SRS were detected via combined EEG and video inspection. EEG signals were first screened to detect spontaneous discharges. Detected discharge events were time-stamped and corresponding video data were reviewed to score motor seizures.

### Materials and Methods—Detailed Description of Experiments—Statistical Analysis.

Statistical tests were conducted using Sigmaplot or Origin software. Data were presented as means and standard error of the mean (SEM) throughout the text and figures. Statistical significance was set at p<0.05. For normally distributed data, group differences were assessed using a Student's t-test or one-way ANOVA followed by a Bonferroni post hoc test. When data were not distributed normally, a Mann-Whitney U test or a nonparametric ANOVA on rank (Kruskal-Wallis) followed by a post hoc test was used for group comparison. A Chi-square or Fisher exact test was used for comparing proportions.

### Materials and Methods—Detailed Description of Experiments—SRS Progression.

SRS development was assessed by the numbers of stimuli needed to induce SRS. For all mice tested (n=42), SRS were observed following 100.4±4.0 to 114.8±4.0 stimuli. Neither seizures nor aberrant hippocampal spikes were observed from the control mice after 60 days of handling manipulations.

### Materials and Methods-Detailed Description of Experiments-EEG Features of SRS.

Concurrent spontaneous discharges in kindled hippocampal and corresponding unstimulated areas were consistently observed from all mice. Most discharges began with low voltage fast (LVF) signals, which were followed by incremental rhythmic spikes and then sustained large-amplitude spikes with simple or complex waveforms. Discharge termination in most cases featured a sudden cessation of spike activity and a subsequent component of signal suppression lasting several seconds. Discharge durations were determined by the time between the LVF onset and the spike cessation. A total of 2,790 SRS events with decipherable EEG signals in both corresponding regional recordings and identifiable motor behaviors in video were collected from 42 mice. Durations of corresponding regional discharges were not significantly different irrespective of severities of associated motor seizures (Student t test or Mann-Whitney U test). However, their waveforms and termination times were different. As local differential recordings through twisted wire bipolar electrodes were used to sample local signals, these differences suggest distinct epileptic activities of different local circuitries. Assessments of regional discharge signals via phase-amplitude coupling and wavelet phase coherence analyses were supportive of this view.

### Materials and Methods—Method.

Results were obtained using the method described with reference to FIG. 1, described in greater detail above.

### Results.

Highly synchronized neurons may be capable of generating a narrow frequency band otherwise, it may become diffused. Based on data analysis results, the chirp may be an inherent characteristic of ictal discharge regardless of being evoked or spontaneous. This activity can be viewed as a representative feature of a large local circuitry, as it may be observed in all studied animals. While electrode locations in each animal may slightly vary, the fundamental feature of chirp may remain more or less consistent.

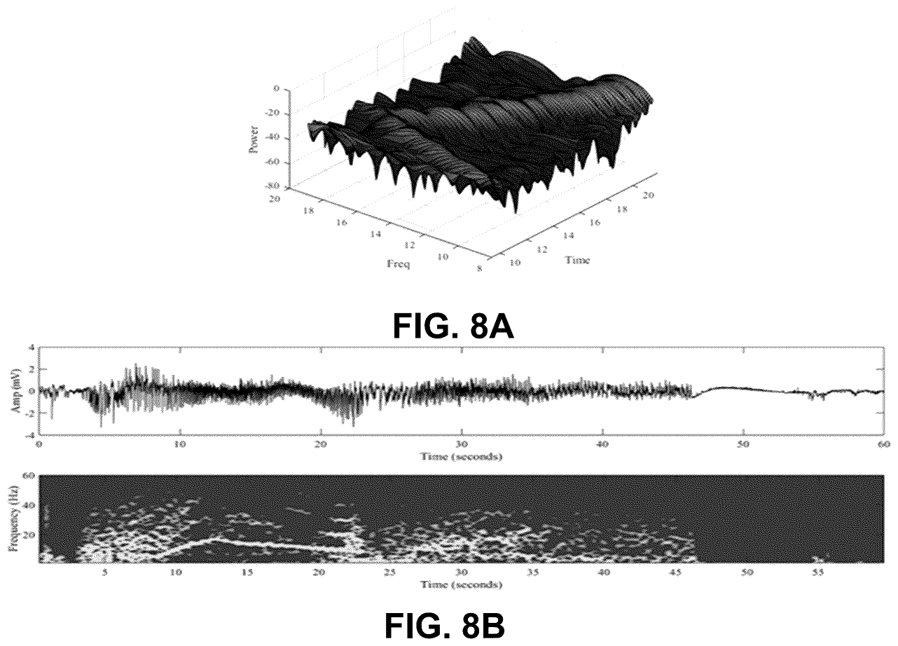

FIG. 8A illustrates a 3D visualization of a sample ictal discharge chirp with asymmetric peak-structured frequency variation over time, according to some embodiments.

FIG. 8B illustrates the recording and 2D time-frequency representation accompanying the visualization of FIG. 8A, according to some embodiments.

FIG. 8A and FIG. 8B shows a trajectory of smoothed power ratios corresponding to an ictal discharge sample, according to some embodiments. In some cases with lower-frequency chirps, visual assessment can fine-tune the pre-defined range of high and low frequency bands.

FIG. 8A shows the evolving frequency pattern of a sample extracted chirp over time. The experimental data analyzed includes ictal event seizures that are evoked (early and late stages), spontaneous (early and late stages), and occurring before, during and after drug application effects.

The data analysis findings may show a progressive evolution of chirp activities, transitioning from its initial formation to the emergence of a fully developed pattern over time. Furthermore, there may be observations of recurring, distinct chirp patterns occurring in a cyclic manner. FIG. 4 shows different spectro-temporal patterns of ictal chirps.

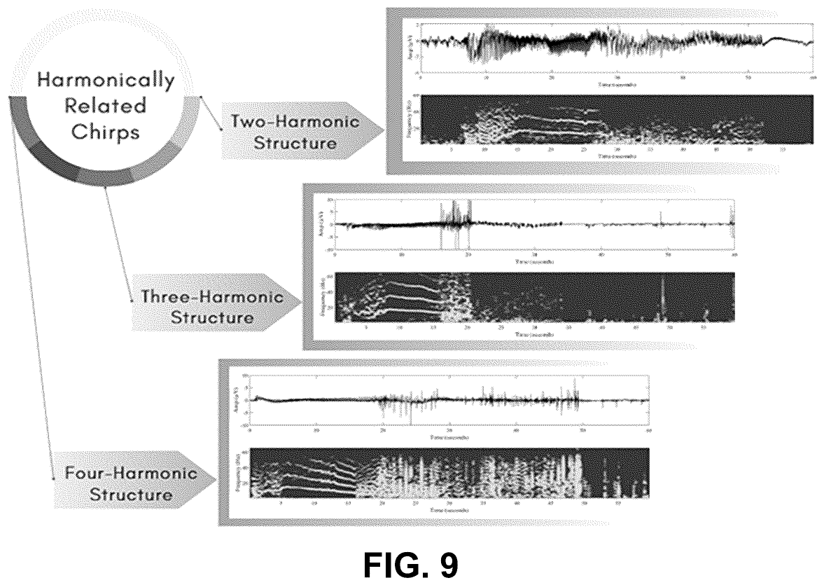

Within certain ictal discharges, it may be observed that the frequencies of individual chirps exhibit a harmonic relationship (see, for example, FIG. 9). The harmonically related chirps may originate within the same structure since otherwise, if they originated from different structural regions, there may be some noticeable delay.

FIG. 9 illustrates chirps exhibiting harmonic relationships, according to some embodiments.

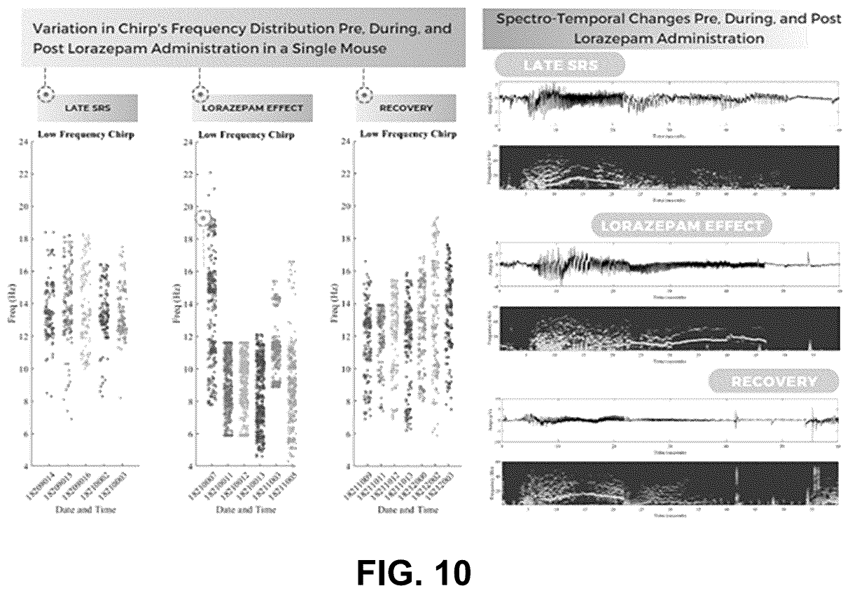

Lorazepam can be used as an anti-seizure medication. While it may have strong peripheral action and may be well-established as an effective agent for relaxing peripheral muscles and mitigating convulsive behavior, it may or may not suppress ictal discharges. Lorazepam may act as a GABAA enhancer. Enhancing GABAA network activity is necessary for the ongoing rhythmic signal. Without a minimum level of GABAergic activity, the rhythm may not be observed. Therefore, increasing GABAA activity may be expected to enhance the rhythmic components. Thus, the GABAA enhancer may be likely to boost the intensity and duration of a chirp (see, for example, FIG. 10).

FIG. 10 illustrates characteristics of a chirp pre, during, and post lorazepam administration, according to some embodiments.

Before the drug is given, the baseline chirp characteristics may provide information about initial neurological state. This baseline may be used as a reference point to compare against the changes that occur after drug administration. When the drug is administered, it may influence the chirp characteristics in various ways. Monitoring the chirp characteristics after drug washout may provide insights into how the seizure activity returns to its baseline state and whether there are any lasting effects from the drug exposure.

In the presence of lorazepam, the following may be observed. Referring to FIG. 10, as time progresses after washing the drug, a gradual and slight upward shift in chirp's frequency may become noticeable during the extended recovery period. Following the drug washout, it may be presumed that the first chirp still reflects the residual impact of the drug. However, as the recovery period lengthens, the drug's influence may gradually dissipate. Initially, the chirp may be characterized by a confined frequency range, which then broadens as the recovery progresses. According to FIG. 10, as the ictal discharge gradually recovers, continuous recording during the recovery phase may lead to the chirp frequency returning to the baseline level that was present before drug injection. As illustrated in FIG. 10, lorazepam may lead to a lengthening of chirp duration and chirp frequency decrease.

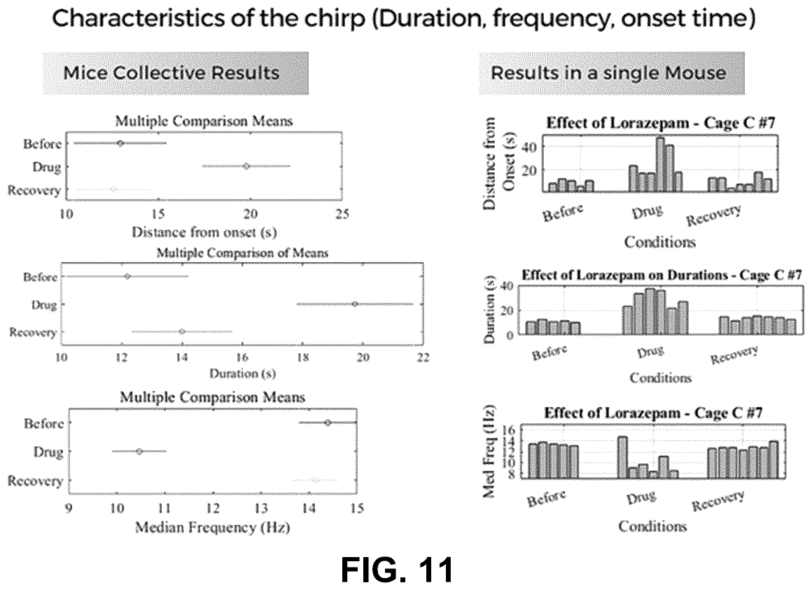

There may be significant differences in the characteristics of chirp before, during, and after lorazepam administration based on analysis of all animals' data. Lorazepam may extend the duration of the chirp, reduce the median frequency of chirp, and prolong the time before chirp onset (see, for example, FIG. 11).

FIG. 11 illustrates further characteristics of a chirp pre, during, and post the drug administration of FIG. 10, according to some embodiments.

Results of this cage may show how lorazepam makes the chirp frequency slower and more stable, and may cause a delay in the chirp's occurrence in the ictal activity.

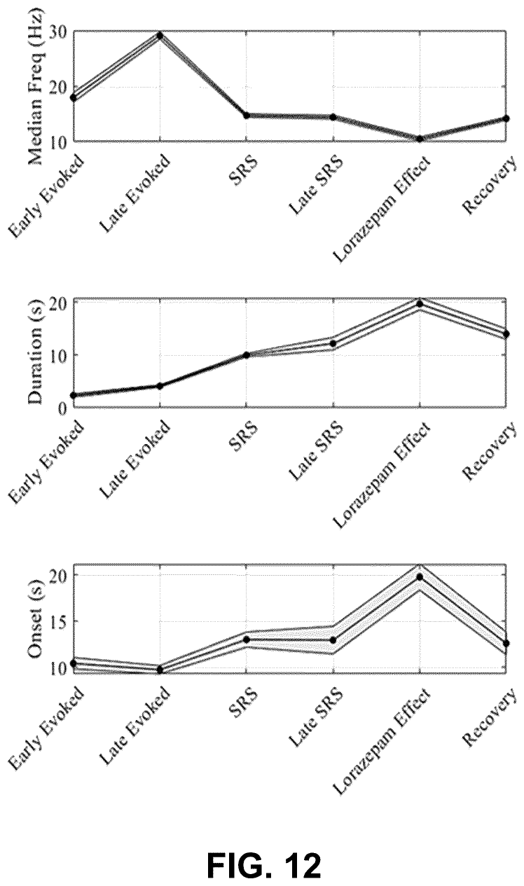

FIG. 12 illustrates changes in mean values and confidence intervals for chirp-like pattern characteristics as epilepsy progresses from the initial evoked state to a drug state, according to some embodiments.

TABLE 1
SRS, and Evoked (early and late) Comparisons

| | Chirp | | Median |
|---|---|---|---|
| | Duration | Chirp Onset | Frequency |
| Comparison | Means (p) | Means (p) | Means (p) |
| Evoked-Early vs. | 0.000025 | 0.37192 | 6.17 × 10⁻¹⁶ |
| Evoked-Late | | | |
| SRS vs. Evoked- | 3.74 × 10⁻¹⁸ | 0.015609 | 0 |
| Late | | | |

Significant differences (p < 0.05), not significant (NS) differences (p ≥ 0.05)

TABLE 2
Pre-, Drug, and Post- Comparisons

| | | Median | |
|---|---|---|---|
| | Duration | Frequency | Chirp Onset |
| Comparison | Means (p) | Means (p) | Means (p) |
| “Before” vs. | 6.46 × 10⁻⁵ | 4.72 × 10⁻¹¹ | 0.00391 |
| “Drug” | | | |
| “Drug” vs. | 0.00091 | 1.96 × 10⁻¹¹ | 0.00077 |
| “Recovery” | | | |
| “Before” vs. | 0.4782 (NS) | 0.84473 (NS) | 0.98195 (NS) |
| “Recovery” | | | |

Significant differences (p < 0.05), not significant (NS) differences (p ≥ 0.05)

FIG. 12 may show the temporal evolution of mean values and their corresponding standard errors for characteristics of chirp-like patterns throughout different stages of epilepsy ictal events, spanning from the initial evoked to late evoked to spontaneous and a drug state. These mean values and their associated standard errors can be obtained by conducting pairwise comparisons using a multiple comparison test to determine the states with statistically significant differences in means. According to statistical analysis, there may be statistically significant difference between early and late evoked ictal states. Evoked discharges occur artificially due to stimulation, which may be why their chirp's frequency is higher compared to spontaneous ictal states (see frequency plot in FIG. 12). In late evoked discharges, the chirps may initiate earlier (see onset plot in FIG. 12).

There may be a mechanism through which neurons can synchronize and produce narrow-band activity, and this process starts earlier after many stimulations (i.e., late evoked states). Initially, it may take longer to observe this chirp because, at the early stimulation stage, neuronal recruitment may be insufficient. To allow the narrow-band activity to occur, a significant amount of wide-band activity may be required. As more stimulations occur, the system may become more facilitative in generating this activity. Artificial stimulation may induce evoked discharges, prompting the system to synchronize, causing these chirps to possibly commence earlier. By enforcing synchronization, the process may be expedited compared to the natural, spontaneous synchronization, which typically takes more time to occur.

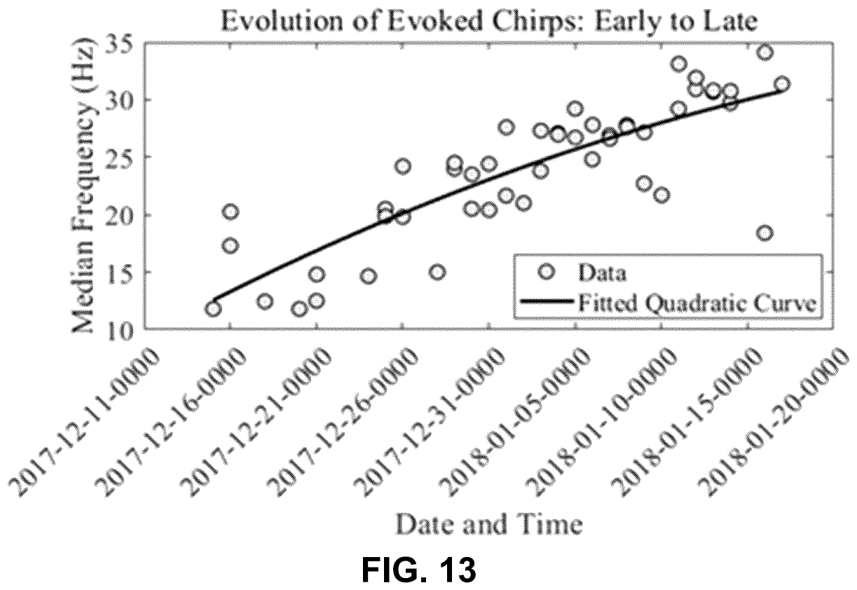

Looking more closely at the changes in chirp median frequency from early to late evoked discharge, there may be a consistent increase over time with a relatively stable rate of change. That is, with increased stimulation, the higher frequency band may rise (see, for example, FIG. 13).

FIG. 13 illustrates changes in chirp median frequency from early to late evoked discharge, according to some embodiments.

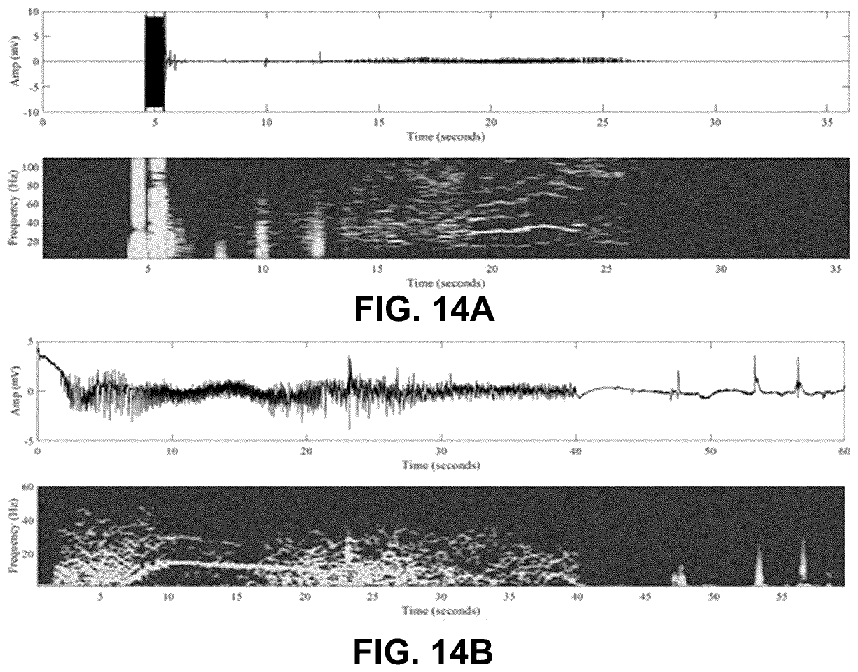

FIG. 14A illustrates the temporal relationship between chirp occurrence and ictal termination for an evoked discharge, according to some embodiments.

FIG. 14B illustrates the temporal relationship between chirp occurrence and ictal termination for a spontaneous recurrent seizure ictal discharge, according to some embodiments.

Considering the phases of a seizure-initiation, maintenance and termination, differences between evoked and spontaneous ictal events may be shown in FIG. 14A and FIG. 14B. For evoked discharges, the chirp may occur just prior to termination. For spontaneous ictal events, chirps may occur clearly within the maintenance phase (middle). In other words, evoked discharges may terminate abruptly following the chirp, while spontaneous ones may do so less distinctly. It appears that this chirp may contribute to the termination of seizure in the early stages.

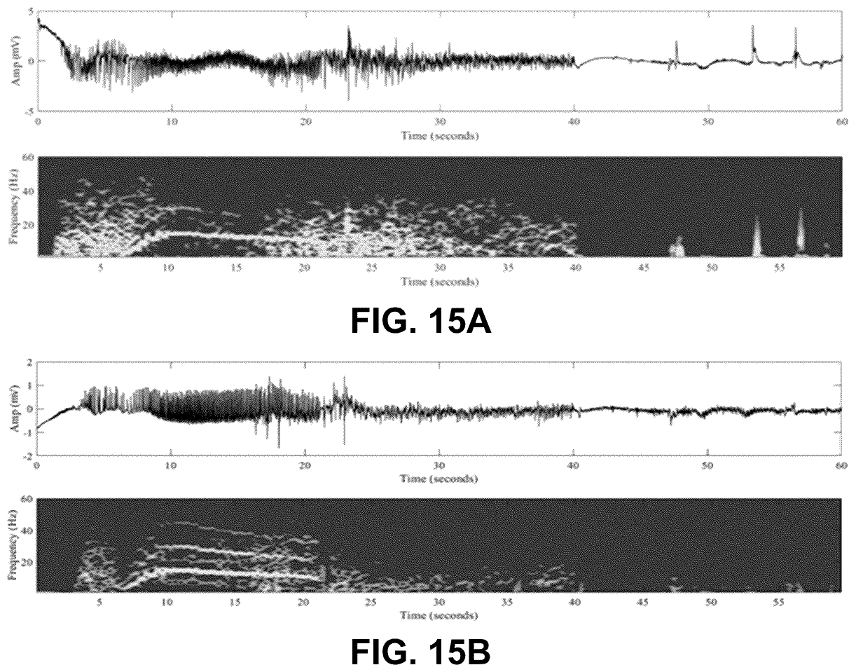

Simultaneously, comparable chirp patterns may be detected in different brain regions within the same animal during an episode of spontaneous recurrent seizure (see, for example, FIG. 15A and FIG. 15B).

FIG. 15A illustrates concurrent chirp patterns during spontaneous recurrent seizure from the ictal discharge in the hippocampus, according to some embodiments.

FIG. 15B illustrates concurrent chirp patterns during spontaneous recurrent seizure from the ictal discharge in the piriform cortex, according to some embodiments.

### Computing Implementation Details

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

For example, and without limitation, the computing device may be a server, network appliance, set-top box, embedded device, computer expansion module, personal computer, laptop, personal data assistant, cellular telephone, smartphone device, UMPC tablets, video display terminal, gaming console, electronic reading device, and wireless hypermedia device or any other computing device capable of being configured to carry out the methods described herein

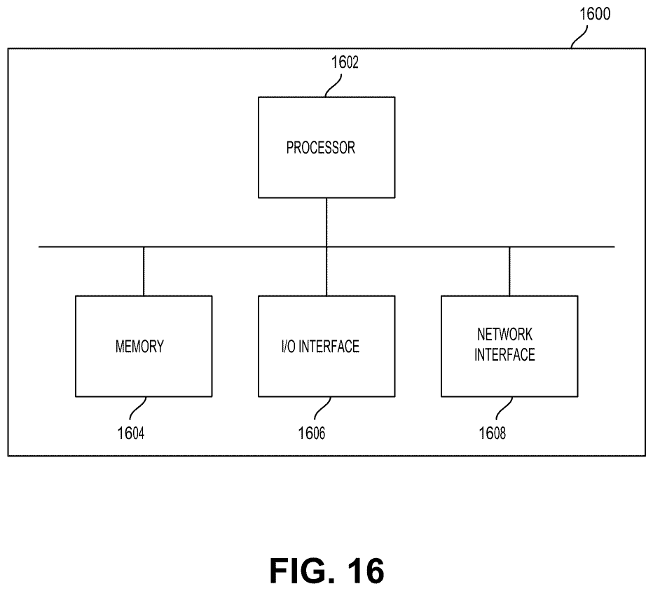

FIG. 16 illustrates a schematic diagram of computing device 1600, according to some embodiments. As depicted, computing device 1600 includes at least one processor 1602, memory 1604, at least one I/O interface 1606, and at least one network interface 1608.

For simplicity only one computing device 1600 is shown but the system may include more computing devices 1600 operable by users to access remote network resources and exchange data. The computing devices 1600 may be the same or different types of devices. The computing device 1600 may include at least one processor, a data storage device (including volatile memory or non-volatile memory or other data storage elements or a combination thereof), and at least one communication interface. The computing device components may be connected in various ways including directly coupled, indirectly coupled via a network, and distributed over a wide geographic area and connected via a network (which may be referred to as “cloud computing”).

Each processor 1602 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof.

Memory 1604 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 1606 enables computing device XX to interconnect with one or more input devices, such as a keyboard, mouse, camera, touch screen and a microphone, or with one or more output devices such as a display screen and a speaker.

Each network interface 1608 enables computing device XX to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

Computing device 1600 is operable to register and authenticate users (using a login, unique identifier, and password for example) prior to providing access to applications, a local network, network resources, other networks and network security devices. Computing devices 1600 may serve one user or multiple users.

The computing device 1600 may be operable to provide the epilepsy monitoring unit 600 of FIG. 6.

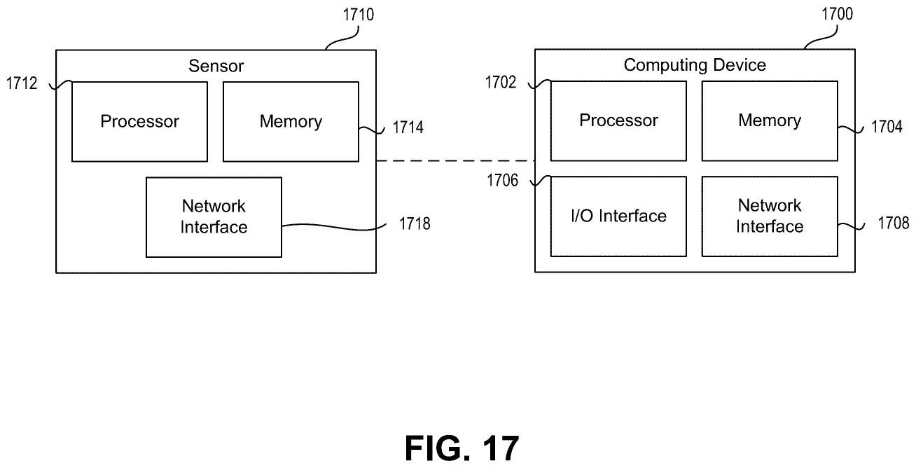

FIG. 17 illustrates a schematic diagram of a system with a computing device 1700 and a sensor 1710, according to some embodiments. For example, the system of computing device 1700 and sensor(s) 1710 can implement a monitoring unit.

As depicted, computing device 1700 includes at least one processor 1702, memory 1704, at least one I/O interface 1706, and at least one network interface 1708.

The computing device 1700 and its components (the processor 1702, the memory 1704, the I/O interface 1706, and the network interface 1708) each share the same general description and variations thereon as described for computing device 1600 and its components (the processor 1602, the memory 1604, the I/O interface 1606, and the network interface 1608) of FIG. 16.

The sensor 1710 may be a sensor configured to receive bio-signals from a user. In some embodiments, the sensor 1710 may comprise one or more electrodes configured to receive EEG signals from a patient. In some embodiments, the sensor 1710 may comprise another sensor modality capable of sensing EEG signals. In some embodiments, 1710 may comprise sensors configured to sense other signal modalities in which relevant chirps may be inferred or from which relevant chirps may be extracted.

In some embodiments, the sensor 1710 may act as a bare signal sensor and transmitter for the computing device 1700. In some embodiments, the sensor 1710 may be indirectly coupled to the computing device 1700. In some embodiments, the sensor may be integrally built into the computing device 1700.

In some embodiments, the sensor 1710 may optionally comprise any one of at least one processor 1712, memory 1714, and at least one network interface 1718. The processor 1712, the memory 1714, and the network interface 1718 may each share the same general description and variations thereon as described for the processor 1602, the memory 1604, and the network interface 1608 of FIG. 16

In particular, the processor 1712 and memory 1714 may be useful to orchestrate the sensor 1710 functions such as sensing any bio-signals and transmitting them to the computing device 1700 (e.g., through the network interface 1718). The processor 1712 may further be configured to carry out pre-processing on the signals received from the patient. For example, before any signals are sent to the computing device 1700 for analysis, they may be processed to, for example, smooth the data, discard invalid readings, filter the data, or carry out any other pre-processing. In some embodiments, the processor 1712 may be configured to carry out some portions of the signal analysis. For example, the processor 1712 may determine onset and offset times for ictal-related chirps before sending the signal and the onset and office times to the computing device 1700. In such embodiments, the computing device 1700 may not need to carry out that same determination. The sensor 1710 may further be configured to carry out any other steps of the analysis as well.

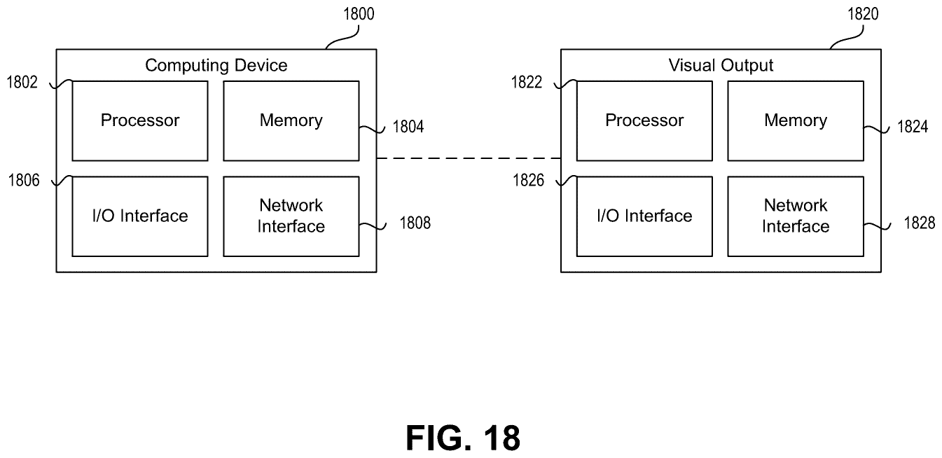

FIG. 18 illustrates a schematic diagram of a system with a computing device 1800 and a visual output 1820, according to some embodiments. For example, visual output 1820 include a display device (e.g. I/O interface) to display data related to detected patterns, morphology, and other chirp related data.

As depicted, computing device 1800 includes at least one processor 1802, memory 1804, at least one I/O interface 1806, and at least one network interface 1808.

The computing device 1800 and its components (the processor 1802, the memory 1804, the I/O interface 1806, and the network interface 1808) each share the same general description and variations thereon as described for computing device 1600 and its components (the processor 1602, the memory 1604, the I/O interface 1606, and the network interface 1608) of FIG. 16.

The visual output 1820 may be an output device to provide an interface, for example, for use by a physician. The interface can have a visual representation of the chirp so that they might be able to assess it by visual inspection. For example, the visual output 1820 may comprise, for example, a display screen to illustrate, for example, visual effects of the chirp morphology. The physician (or any user) may be able to ascertain particular information about the ictal event based on the visual information alone (e.g., from the morphological type of the chirp). In some embodiments, other information may be output (e.g., key features of the chirp, other visualizations, comparators, historic data, etc.).

In some embodiments, the visual output 1820 may optionally comprise any one of at least one processor 1822, memory 1824, at least one I/O interface 1826, and at least one network interface 1828. The processor 1822, the memory 1824, at least one I/O interface 1826, and the network interface 1828 may each share the same general description and variations thereon as described for the processor 1602, the memory 1604, the at least one I/O interface 1606, and the network interface 1608 of FIG. 16.

In particular, the processor 1822 may be configured to orchestrate the visualization of the chirp data received from a computing device 1800 to provide, for example, a GUI for easy visual assessment. Memory 1824 may be configured to store instructions for visualization applications. The memory 1824 may be able to store data (e.g., exemplar data to which the patient's data may be compared). The visual output 1820 may be configured to visualize both the patient's data and exemplar data. In some embodiments, the visual output 1820 may be configured to access past patient data (e.g., from memory 1824 or memory 1804 or a server) to visualize historic data as compared to current data. In some embodiments, signal charts may be displayed (which may aid a physician to diagnose, monitor, investigate, or treat just by looking at the display). The I/O Interface 1826 may be configured to receive instructions from an input device and modify the visualization on the screen (or other output device). For example, a physician may be able to actuate a touch screen to move, scroll, spin, or otherwise manipulate one or more visualizations being displayed. The network interface 1828 may be configured to communicate with the computing device 1800 or other system components (e.g., a server).

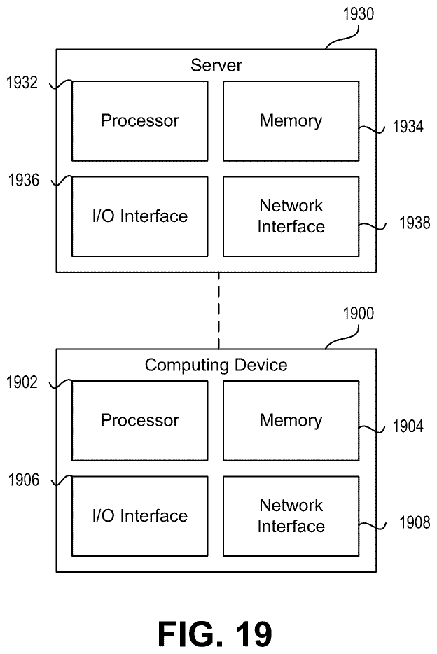

FIG. 19 illustrates a schematic diagram of a system with a computing device 1900 and a server 1930, according to some embodiments.

As depicted, computing device 1900 includes at least one processor 1902, memory 1904, at least one I/O interface 1906, and at least one network interface 1908. The computing device 1900 and its components (the processor 1902, the memory 1904, the I/O interface 1906, and the network interface 1908) each share the same general description and variations thereon as described for computing device 1600 and its components (the processor 1602, the memory 1604, the I/O interface 1606, and the network interface 1608) of FIG. 16.

The server 1930 may act as a store for patient data or for updated models and systems used in analysis. For example, the computing device 1900 may be configured to pull the most up-to-date models from the server 1930. As a further example, the computing device 1900 may be configured to carry out some processing on the data and send the rest to the server 1930 to be processed. Models may be stored on the server 1930 so that a plurality of computing devices 1900 are configured to pull the most recent models from the server 1930 (rather than manually uploading the updated models to each of the computing devices 1900). In some embodiments, the computing device 1900 may deidentify patient information before sending it to the server 1930 for any processing that may occur at the server 1930 to ensure that patient privacy is maintained in the event of the server 1930 being hacked.

In some embodiments, the server 1930 may optionally comprise any one of at least one processor 1932, memory 1934, at least one I/O interface 1936, and at least one network interface 1938. The processor 1932, the memory 1934, and the network interface 1938 may each share the same general description and variations thereon as described for the processor 1602, the memory 1604, the at least one I/O interface 1606, and the network interface 1608 of FIG. 16.

In particular, the processor 1932 may be configured to carry out processes to operate and maintain the server 1930. In some embodiments, the processor 1932 may be configured to analyze chirps as described herein. The memory 1934 may store instructions to carry out any processes performed by the server 1930. In some embodiments, the memory 1934 may be configured to store patient data (for example, biographical information, medical history, demographic information, prior analysis, physician notes, etc.). In some embodiments, there may be a first server 1930 which stores updated models for data analysis and a second server 1930 to store patient information. These servers may or may not be in direct communication. The I/O Interface 1936 may support users inputting information directly into the server 1930. The network interface 1938 may support communication between the server 1930 and a computing device 1900. In some embodiments, the network interface 1938 may support communication between the server 1930 and other system components such as sensors and/or visual outputs.

FIG. 16, FIG. 17, FIG. 18, and FIG. 19 each show system configurations that may make use of the systems and methods described herein. The examples shown are not to be construed as examples which limit the possible meriad of system configurations. For example, a system may include a computing device 1600 of FIG. 16, a sensor 1710 of FIG. 17, a visual output 1820 of FIG. 18, and a server 1930 of FIG. 19. Further variations are conceived. Each of these components may carry out one or more of the process steps described in this application. For example, any or all of these systems or component parts thereof may be operable to provide the epilepsy monitoring unit 600 of FIG. 6.

### Implementation Details

The foregoing discussion provides many example embodiments. Although each embodiment represents a single combination of inventive elements, other examples may include all possible combinations of the disclosed elements. Thus if one embodiment comprises elements A, B, and C, and a second embodiment comprises elements B and D, other remaining combinations of A, B, C, or D, may also be used.

The term “connected” or “coupled to” may include both direct coupling (in which two elements that are coupled to each other contact each other) and indirect coupling (in which at least one additional element is located between the two elements).

Although the embodiments have been described in detail, it should be understood that various changes, substitutions and alterations can be made herein without departing from the scope as defined by the appended claims.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps

As can be understood, the examples described above and illustrated are intended to be exemplary only. The scope is indicated by the appended claims.

What is claimed is:

1. A computer implemented method comprising:
identifying ictal-related chirp patterns from recordings of electrophysiological signals by determining onset and offset times of the ictal-related chirp patterns;
characterizing the ictal-related chirp patterns;
classifying spectro-temporal morphology of the ictal-related chirp patterns; and
transmitting or storing data for the ictal-related chirp patterns.

2. The method of claim 1 wherein determining the onset and offset times of the Ictal-related chirp patterns comprises:
computing a smoothed power ratio trajectory of defined high to low frequency bands over time;
applying a threshold to the smoothed power ratio trajectory;
identifying instances where the power ratio trajectory exceeds the threshold;
confirming that the identified event is statistically significant;
defining chirp onset as the moment when the power ratio trajectory significantly exceeds threshold;
defining chirp offset as the moment when the power ratio trajectory significantly falls below threshold; and
fine-tuning the defined range of high and low frequency bands.

3. The method of claim 2 wherein high-frequency range is set between 10 Hz and 22 Hz, and low-frequency range is set between 1 Hz and 10 Hz.

4. The method of claim 1 further comprising tracking chirp morphology by extracting a dominant frequency ridge curve from a time-frequency representation using a penalized forward-backward greedy algorithm between onset and offset times of the ictal-related chirp patterns with customizable parameters to penalize frequency changes and determine a number of ridges.

5. The method of claim 1 wherein characterizing the ictal-related chirp patterns comprises determining temporal and spectral characteristics by analyzing features comprising duration of chirp, median frequency of chirp, and chirp onset.

6. The method of claim 1 wherein classifying the spectro-temporal morphology of the ictal-related chirp patterns comprises categorizing the spectro-temporal morphology into a type based on the evolution of frequency characteristics over time.

7. The method of claim 1 wherein generating the data about the chirp morphology comprises statistical testing including one-way Analysis of Variance (ANOVA) and Tukey's test to identify specific pairs of conditions displaying significant differences.

8. The method of claim 4 further comprising extracting the time-frequency representation by spectrogram calculation of segmented the electrophysiological signal, applying windowing functions, and performing Discrete Fourier Transform to reveal spectral evolution of the electrophysiological signal over time.

9. The method of claim 1 wherein determining the onset and offset of chirp comprises analyzing a power ratio of specific frequency bands and identifying chirp events based on a threshold.

10. The method of claim 1 further comprising generating the data about the chirp morphology by determining characteristics of chirp-like activities that reflect different subject states comprising early evoked discharge, late evoked discharge, spontaneous recurrent seizure, and a drug state.

11. The method of claim 1 wherein the electrophysiological signals are selected from the group of EEG, intracranial EEG, and Local Field Potentials signals.

12. The method of claim 1 wherein a chirp is an electrophysiological signal whose frequency changes over time.

13. The method of claim 1, further comprising:
characterizing at least one of progression of a disease and effect of a drug based in part on the ictal-related chirp patterns.

14. The method of claim 1, further comprising:
elucidating a mechanism of action of a drug based in part on the ictal-related chirp patterns.

15. The method of claim 1 wherein classifying the spectro-temporal morphology of the ictal-related chirp patterns comprises categorizing the spectro-temporal morphology into one of Type 1-5, wherein:
Type 1 exhibits a decline in frequency with a semi-linear trend;
Type 2 exhibits a stepped increment in frequency over time;
Type 3 exhibits asymmetric peak-structured frequency variations;
Type 4 exhibits symmetric peak-structured frequency changes; and
Type 5 exhibits a rapid initial increase in frequency followed by a stable frequency profile over time.

16. The method of claim 1 wherein classifying the spectro-temporal morphology of the ictal-related chirp patterns comprises determining that the spectro-temporal morphology of the ictal-related chirp patterns occur in a cyclic manner.

17. The method of claim 1, further comprising:
determining whether the ictal-related chirp patterns arise from a spontaneous ictal event or an evoked discharged based on timing of the chirp within the ictal event.

18. The method of claim 1, further comprising:
verifying the spectro-temporal morphology classification of the ictal-related chirp patterns by comparing the spectro-temporal morphology classification with a spectro-temporal morphology classification of ictal-related chirp patterns arising from another brain region.

19. The method of claim 1 further comprising generating visual elements related to the data for the ictal-related chirp patterns and chirp morphology, and displaying, at an interface, the visual elements related to the data for the ictal-related chirp patterns and chirp morphology.

20. The method of claim 1 further comprising transmitting the data for the ictal-related chirp patterns and visual elements related to the data for the ictal-related chirp patterns to an interface of a computing device.

21. An epilepsy monitoring unit for diagnosis, investigation or treatment of seizures comprising:
one or more sensors for capturing electrophysiological signals;
a processing system that includes one or more processors and one or more memories coupled with the one or more processors, the one or more memories storing recordings of electrophysiological signals;
the processing system configured to cause monitoring unit to:
identify ictal-related chirp patterns from recordings of electrophysiological signals by determining onset and offset times of the Ictal-related chirp patterns;
characterize the ictal-related chirp patterns; and
classify chirp morphology.

22. A computer implemented method for electrophysiological signals, the method comprising:
extracting a neuromarker from neural activities recorded as electrophysiological signals; and
characterizing ictal-related patterns in the electrophysiological signals during ictal discharge,
wherein the neuromarker is for assessment of progression of a seizure or efficacy of anti-seizure pharmacological agents.

23. The method of claim 22 wherein the neuromarker comprises a plurality of features derived from chirp-like patterns in the electrophysiological signals, the plurality of features comprising morphology of a chirp, duration of the chirp, onset time, frequency band and power distribution.

24. The method of claim 22 further comprising generating neuromarker-based evaluations or developing a personalized treatment plan to improve seizure control.
